(19) United States
(12) Patent Application Publication
(10) Pub. No.: US 2004/0220221 A1
(43) Pub. Date: Nov. 4, 2004

(54) Treatment of degenerative diseases

(76) Inventors: Helen Frances Baker, Essex (GB); Robin Mark Bannister, Essex (GB); Alan Rothaul, Essex (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 32606-6669, US

(21) Appl. No.: 10/345,530

(22) Filed: Jan. 16, 2003

(30) Foreign Application Priority Data
Jan. 17, 2002 (GB) 0201025.4

(51) Int. Cl. A61K 31/4745; A61K 31/47

(52) U.S. Cl. 514/313; 514/305

(57) ABSTRACT

According to the subject invention, antimalarial drugs (including salts or isomers thereof) may be used to treat degenerative disease. Antimalarial drugs include mefloquine, the enantiomers of mefloquine, hydroxychloroquine, the enantiomers of hydroxychloroquine, chloroquine, primaquine and quinine, and also their metabolites.

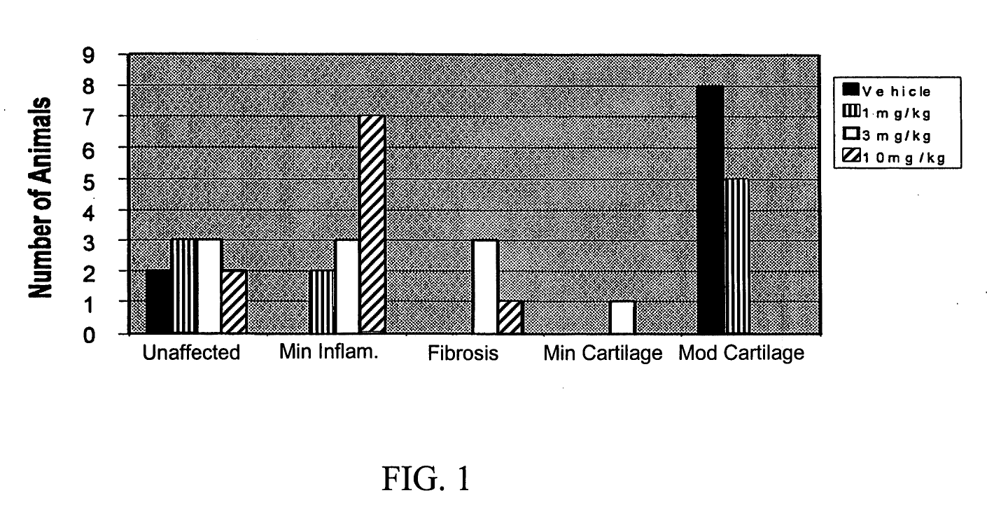

### FIELD OF THE INVENTION

The present invention relates to the treatment of degenerative diseases.

### BACKGROUND OF THE INVENTION

Cytokines belong to a large group of polypeptide- or glycopeptide-signalling molecules that act, at extremely low concentrations, as regulators of cell growth and essential mediators of inflammation and immune reactions. The production and functions of cytokines are tightly regulated by cytokines themselves and by several other factors. Most cytokines act locally and are implicated in a number of inflammatory conditions. These include rheumatoid arthritis (RA), osteoarthritis (OA), psoriatic arthritis, psoriasis, ulcerative colitis and Crohn's disease.

The antimalarial compounds chloroquine and hydroxychloroquine are known as broadly active, modestly potent inhibitors of cytokines. Such antimalarial agents have become important disease-modifying antirheumatic agents (DMARD) in the second line treatment of rheumatoid arthritis and other inflammatory disorders. Other agents in this class include gold, penicillamine, methotrexate and cyclosporins, all of which have potent activity. However, the utility of these latter drugs for the treatment of a chronic disease such as rheumatoid arthritis is limited by serious side-effects. The antimalarial agents in the DMARD class are recognised as having a more moderate side-effect profile, while possibly lacking the potency of some of the other agents. However, there is still concern about the ocular side-effects of both chloroquine and hydroxychloroquine. Thus, it may be postulated that a drug for the treatment of arthritis that possesses an improved efficacy versus side-effect profile over hydroxychloroquine, the most significant antimalarial drug in the DMARD class, would be of significant clinical potential.

In terms of antimalarial potency, mefloquine is one of the most effective drugs indicated for both prophylaxis and treatment and has particular utility for use in chloroquine-resistant malaria. Chloroquine has been the mainstay of antimalarial treatment and prophylaxis, but the emergence of chloroquine resistance in Plasmodium falciparum, the most lethal strain, has started to limit its utility. Thus mefloquine has emerged as the preferred compound for the prophylaxis and treatment of malignant malaria.

Mefloquine enantiomers have been evaluated in animal models for efficacy against Plasmodium species. These studies concluded that there was no difference in antimalarial potency of the enantiomers.

PCT/GB01/03924 discloses that (+)-mefloquine has particular utility in the treatment of inflammatory disorders, especially rheumatoid arthritis and osteoarthritis.

### SUMMARY OF THE INVENTION

Surprisingly, it has been shown that antimalarials have utility in the treatment of degenerative diseases such as osteoarthritis. According to the invention, antimalarial drugs (including salts or isomers thereof) may be used to treat degenerative disease. Antimalarial drugs include mefloquine, the enantiomers of mefloquine, hydroxychloroquine, the enantiomers of hydroxychloroquine, chloroquine, primaquine and quinine, and also their metabolites.

### DESCRIPTION OF PREFERRED EMBODIMENTS

Degenerative disease includes degenerative diseases of the joints/bones (e.g. osteoarthritis, periodontal disease, malunion following fracture, Paget's disease), of the pulmonary system (e.g. cystic fibrosis, COPD, bronchiectasis, lung disorders associated with scarring), of the neurological system (e.g. Parkinson's, Alzheimer's, multiple sclerosis, stroke), of the skin/eye (e.g. non-healing ulcers, corneal ulcers, apthous ulcers) and primary pulmonary hypertension.

Antimalarial drugs may be used alone or they may also be used in combination with other therapeutics that are normally used in the treatment of these diseases. These agents can be dosed separately or formulated into the same products, ensuring that the release profiles are manipulated to enable the drugs to be dosed appropriately.

The drug or drugs may be useful in indications where no anti-inflammatory effect is desired and indeed it may be useful that there is no interference with the physiological inflammatory response.

The drug may be delivered in various formulations, oral, intravenous, intra-articular, topical, rectal, vaginal, inhalation, intranasal, intracrevicular, intravitreol, retro-orbital. Suitable dosage forms include conventional release, controlled release, enteric-coated and timed release.

Antimalarial drugs may be conventionally formulated or formulated in a controlled release product to give a once daily administration, or in an enteric-coated formulation to provide protection of the drug from stomach acid.

For use in the invention, the active agent may be formulated, e.g. together with a carrier, excipient or diluent, and administered, by procedures that are known in the art, including those already proposed for such compounds. Suitable compositions will depend on the intended route of administration.

The amount of the agent that should be administered can readily be determined by the skilled man, taking into account the-usual factors such as the type of patient, the nature of the condition being treated, and the route of administration. The amount of enantiomer may be higher or the same as that for the racemate, or may be modified depending on the co-administration of other drugs.

Depending on the relative activities of the individual enantiomers, it may be preferred to administer a mixture, e.g. racemate, or substantially one enantiomer. The desired enantiomer may be in at least 50%, 70%, 90%, 95% or 99% excess, with respect to any other. The active agent may be used in any active form, e.g. salt or non-salt.

The use of (+)-erythro-mefloquine is preferred. It appears that this compound is particularly useful in providing the desired effect, without tissue destruction, and can be safely administered at a relatively high dosage.

The following description gives evidence on which the invention is based. The model is experimentally induced osteoarthritis in guinea pigs.

Dunkin Hartley guinea pigs(apart from the blank control) were anaesthetised and underwent partial medial meniscectomy in the right hind leg. 3 doses of +mefloquine, (1 mg/kg/day, 3 mg/kg/day and 10 mg/kg/day) were administered orally by gavage to groups of 10 animals as well as a vehicle control. The vehicle was 0.2% methylcellulose and 0.4% Tween 80 in bidistilled water for the mefloquine groups. The administration volume was 2 mL/kg.

The animals were followed for 10 weeks. Terminal blood samples were taken for drug levels. Histology was performed on the joint, which was operated on, and an assessment of the cartilage structure, cell proliferation and loss of proteoglycans was made. The histology was performed by a blinded pathologist. The results are given in the accompanying drawing, and show that +mefloquine prevents the degeneration of cartilage.

1. A method for the treatment of a degenerative disease, which comprises administering to a patient in need thereof an effective amount of an antimalarial drug wherein said administration is oral administration.

2. The method of claim 1, wherein the degenerative disease is of the joints or bones.

3-6. (Canceled)

7. The method of claim 1, wherein the drug is selected from the group consisting of chloroquine, hydroxychloroquine, primaquine or quinine, or a salt or isomer thereof.

8. (Canceled)

9. The method of claim 1, wherein the drug is (+)-mefloquine.
